(19) United States
(12) Patent Application Publication
(10) Pub. No.: US 2022/0008019 A1
(43) Pub. Date: Jan. 13, 2022

(54) Heart Rate Correction Using External Data

(71) Applicant: Owlet Baby Care Inc., Lehi, UT (US)

(72) Inventors: Cory Jones, Orem, UT (US); Tanner Christensen, Provo, UT (US)

(21) Appl. No.: 17/370,325

(22) Filed: Jul. 8, 2021

Related U.S. Application Data
(60) Provisional application No. 63/049,585, filed on Jul. 8, 2020.

(51) Int. Cl. A61B 5/00; A61B 5/024; G16H 50/20

(52) U.S. Cl. CPC A61B 5/7275; A61B 5/02427; A61B 5/7264; A61B 2562/0219; A61B 5/721; A61B 5/7267; G16H 50/20; A61B 5/7257

(57) ABSTRACT

A technology for improving heart rate prediction. In one example, a machine learning model can be trained using photoplethysmogram (PPG) data to classify a signal in a dataset obtained from a generic data source as a heart rate. Thereafter, a PPG dataset generated by a PPG sensor can be input to the machine learning model to classify a PPG signal in the PPG dataset as a predicted heart rate. Accelerometer data generated by an accelerometer can be input to the machine learning model to classify an acceleration signal contained in the accelerometer data as a heart rate, where movement represented by the acceleration signal mimics the heart rate. The PPG signal in the PPG dataset can be identified as corresponding to the acceleration signal that mimics the heart rate, and the PPG signal can be removed to improve the accuracy of the heart rate prediction.

### BACKGROUND

Non-invasive health monitoring devices are increasingly helping people to better monitor their health status. Some consumer wearable devices have incorporated sensors for gathering biometric data, such as a pulse oximeter, which can be used to generate a photoplethysmogram (PPG). A PPG is an optically obtained plethysmogram which can be used to detect blood volume changes in the microvascular bed of living tissue. A PPG can be obtained using a pulse oximeter which illuminates the skin and measures changes in light absorption.

A pulse oximeter monitors the perfusion of blood to the dermis and subcutaneous tissue of the skin. With each cardiac cycle the heart pumps blood to the periphery causing a pressure pulse that distends the arteries and arterioles in the subcutaneous tissue. A change in volume caused by the pressure pulse is detected by illuminating the skin with the light from a light-emitting diode (LED) and then measuring the amount of light either transmitted or reflected to a photodiode where each cardiac cycle appears as a peak.

### SUMMARY

A system is described for improving a heart rate prediction. The system may include at least one processor, a memory device including instructions that, when executed by the at least one processor, cause the system to obtain a PPG dataset generated by a PPG sensor included in a device. The instructions that, when executed by the at least one processor, may cause the system to input the PPG dataset to a machine learning model trained to classify a PPG signal in the PPG dataset as a predicted heart rate, wherein the machine learning model classifies PPG signals contained in the PPG dataset as predicted heart rates. The instructions that, when executed by the at least one processor, may cause the system to obtain accelerometer data generated by an accelerometer included in the device and input the accelerometer data to the machine learning model, which classifies an acceleration signal contained in the accelerometer data as a heart rate, indicating that movement represented by the acceleration signal mimics the heart rate. The instructions that, when executed by the at least one processor, may cause the system to identify the PPG signal in the PPG dataset that corresponds to the acceleration signal that mimics the heart rate, and remove the signal that corresponds to the acceleration signal from consideration and/or from the PPG data to improve accuracy of the heart rate prediction.

A method is described for determining fetal movement. The method may include receiving a PPG dataset generated by a PPG sensor and inputting the PPG dataset to a machine learning model to classify a PPG signal in the PPG dataset as a predicted heart rate, wherein the machine learning model has been trained using PPG training data to classify a signal from a generic data source as a heart rate. The method may include receiving accelerometer data generated by an accelerometer and inputting the accelerometer data to the machine learning model to classify an acceleration signal contained in the accelerometer data as a heart rate, wherein movement represented by the acceleration signal mimics the heart rate. The method may include identifying a heart rate confidence score output by the machine learning model that corresponds to the acceleration signal that mimics the heart rate, and removing that heart rate from consideration to improve accuracy of the true heart rate prediction.

A non-transitory machine-readable storage medium including instructions embodied thereon is provided. The instructions, when executed by at least one processor may receive a PPG dataset generated by a PPG sensor and input the PPG dataset to a machine learning model to classify a PPG signal in the PPG dataset as a heart rate prediction. The instructions, when executed by at least one processor may receive accelerometer data generated by an accelerometer, wherein the accelerometer data corresponds to a time frame of the PPG dataset, and input the accelerometer data to the machine learning model to classify an acceleration signal contained in the accelerometer data as a heart rate, wherein movement represented by the acceleration signal mimics the heart rate. The instructions, when executed by at least one processor may identify the PPG signal in the PPG dataset as corresponding to the acceleration signal that mimics the heart rate and remove the PPG signal that corresponds to the acceleration signal to improve accuracy of the heart rate prediction.

### BRIEF DESCRIPTION OF THE DRAWINGS

FIG. 1A is a flow diagram illustrating an example system and method for correcting a heart rate probability using accelerometer data.

FIG. 1B is a block diagram that illustrates an example device configured to generate a heart rate probability in accordance with one aspect of the technology.

FIG. 2 is a block diagram that illustrates an example architecture of an artificial neural network model configured to output a heart rate probability.

FIGS. 3A-B illustrate flow diagrams for example methods for preprocessing PPG data and accelerometer data.

FIG. 4 is a flow diagram illustrating an example network architecture for an artificial neural network model which incorporates prior heart rate information to generate a heart rate probability.

FIG. 5 is a flow diagram that illustrates an example method to generate heartbeat confidence scores for PPG data and accelerometer data used to select a heart rate probability.

FIG. 6 is a flow diagram that illustrates an example method for improving the accuracy of a heart rate prediction.

FIG. 7 is a block diagram illustrating an example of a computing device that may be used to execute a method for improving the accuracy of a heart rate prediction.

### DETAILED DESCRIPTION

Technologies are described for improving a heart rate prediction generated using a machine learning model, such as an artificial neural network (ANN) model, a decision tree model, a naive Bayes model, or another appropriate machine learning model. In one example configuration, a heart rate prediction can be improved by identifying a motion-based signal in a photoplethysmogram (PPG) signal dataset that mimics a heart rate and the motion-based signal can be excluded as a heart rate candidate to prevent the motion-based signal as being selected as a predicted heart rate. For example, an ANN model can be trained using PPG data to classify a signal from a generic data source (e.g., a PPG sensor, an accelerometer, or other data source) as a predicted heart rate. Because some types of repetitive movements (e.g., running, swinging, bouncing, etc.) can generate motion-based signals, these motion-based signals can be introduced as noise into a PPG dataset. When the PPG dataset is input to the ANN model, a motion-based signal may be mistakenly classified as a predicted heart rate instead of a PPG signal that represents a true heart rate.

In order to prevent a motion-based signal from being classified as a predicted heart rate, the motion-based signal can be identified using external data generated by an accelerometer, and the motion-based signal can be excluded as a heart rate candidate. For example, in addition to classifying a PPG signal as a predicted heart rate, the ANN model can classify an acceleration signal included in accelerometer data as corresponding to a heart rate. As an example, accelerometer data generated by an accelerometer can be input to the ANN model, and the ANN model can determine whether an acceleration signal contained in the accelerometer data corresponds to one or more characteristics of a heart rate. In the case that the ANN model classifies the acceleration signal as a heart rate, a motion-based PPG signal (e.g., noise generated by movement) that corresponds to the acceleration signal can be identified in the PPG signal dataset, and the motion-based PPG signal can be removed to prevent the motion-based signal from being selected as the predicted heart rate, thereby improving the probability that a PPG signal that matches the true heart rate is selected. In one example, the motion-based PPG signal identified in the PPG dataset can be removed from consideration as a true heart rate (an actual heart rate of a subject) in order to allow the true heart rate signal in the PPG dataset to be selected as a heart rate prediction. For example, the output of the ANN model can be a heart rate confidence score that indicates whether a motion based signal corresponds to a heart rate, and the corresponding signal in the PPG dataset can be identified and excluded as a heart rate candidate. In another example, it is believed that the motion-based PPG signal can be removed from the PPG signal dataset, such that an actual heart rate signal remains in the PPG signal dataset and the actual heart rate signal can be selected as a heart rate prediction.

In one example configuration, the ANN model can include: a first series of convolution layers used to identify a PPG signal in the PPG data and remove artifacts contained in the PPG data, a fast Fourier transform (FFT) layer used to identify PPG frequencies in the PPG data, and a dense layer used to decode a heart rate value from the PPG frequencies. After training the ANN model, a PPG signal can be obtained from a PPG sensor (e.g., a pulse oximeter) and PPG data representing the PPG signal can be input to the ANN model which outputs a heart rate prediction that represents a heart rate extracted from the PPG signal. As will be appreciated, the ANN model can include additional components used to predict a heart rate.

The network architecture of the ANN model provides improvements in the accuracy of heart rate predictions obtained from a PPG signal over previous methods for computing a heart rate from a PPG signal. In particular, the accuracy of heart rate predictions output by the ANN model is improved by placing an FFT layer after a first series of convolution layers and providing the output of the FFT layer to a dense layer of the ANN model. Placement of the FFT layer in this way improves the accuracy of heart rate predictions by using the FFT layer to identify fundamental and harmonic frequencies of a PPG signal, thereby reducing a number of parameters provided to the dense layer of the ANN model.

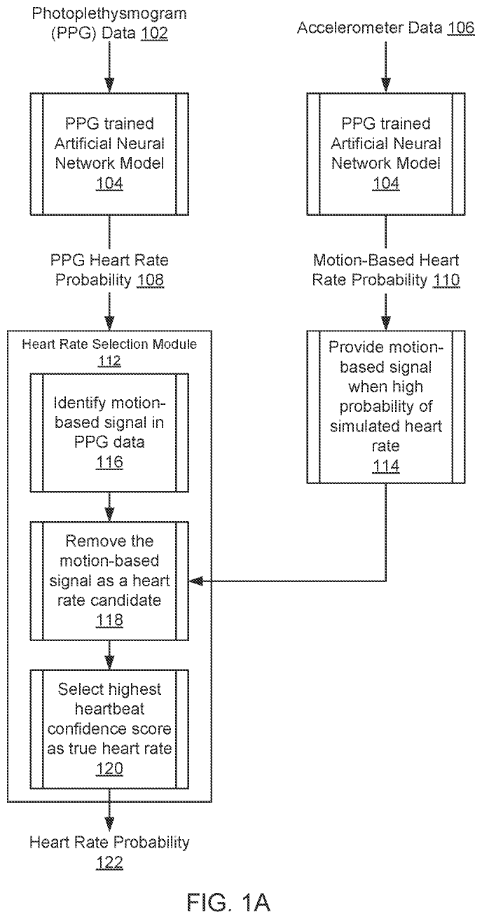

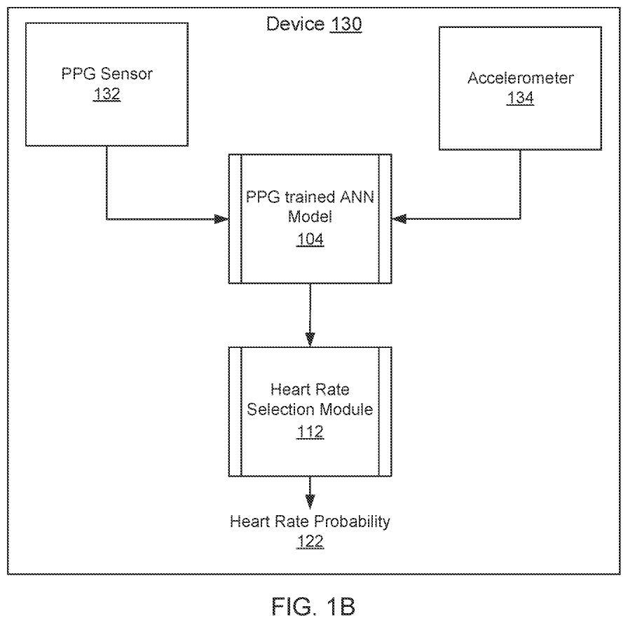

To further describe the present technology, examples are now provided with reference to the figures. FIG. 1A is a flow diagram illustrating a high-level example of a processing system and method for improving a heart rate probability using accelerometer data. The processing system includes an ANN model 104 trained using PPG data to classify a signal obtained from, or generated by, a generic data source (PPG sensor, accelerometer, etc.) as a predicted heart rate. As used herein, “generic data source” refers to any source that can generate a signal. In the examples illustrated herein, the sources of a signal include a PPG sensor and an accelerometer. However, the source of a signal is not limited to a PPG sensor and an accelerometer, and can include other data sources.

The ANN model 104, in one example, is an end-to-end neural network. As described in more detail later, the architecture of the ANN model 104 can include a series of convolution layers followed by an FFT layer and a dense decoding layer. The ANN model 104 can be trained to classify a signal as a predicted heart rate using a training dataset of PPG data. After training, the ANN model 104 can be used to classify signals contained in any type of data that correspond to characteristics of a heart rate. For example, the ANN Model 104 can be used to classify signals contained in PPG data 102, and signals contained in accelerometer data 106, as predicted heart rates. A motion-based signal classified as a predicted heart rate can be compared to PPG signal classified heart rates to determine whether the motion-based signal mimics any of the PPG signal classified heart rates. Any motion-based signals identified as corresponding to PPG signal classified heart rates can be removed (e.g., removed from consideration and/or from the PPG data 102) to improve the accuracy of a heart rate probability generated by the ANN model 104. For example, repetitive movements, such as running, swinging, bouncing, and the like can generate a motion-based signal that is introduced as noise into PPG data 102. When the PPG data 102 is input to the ANN model 104, the motion-based signal may be mistakenly classified as a heart rate probability 122 instead of a PPG signal that represents a true heart rate.

A heart rate selection module 112 can be configured to improve the accuracy of a heart rate prediction output by the ANN model 104 by determining whether a PPG heart rate probability 108 output by the ANN model 104 contains a motion-based signal which could potentially be incorrectly selected as a heart rate probability 122. In one example, as shown in FIG. 1B, PPG data 102 and accelerometer data 106 can be obtained from a PPG sensor 132 and an accelerometer 134 included in a device 130. The device 130 can be a wearable device, such as a wrist worn device (e.g., fitness tracker, activity monitor, smartwatch, etc.), sock (e.g., smart sock), belly band, or any other type of wearable device that includes a PPG sensor 132 and accelerometer 134. A PPG sensor 132 can comprise a light source (e.g., a light emitting diode (LED)) and a light sensor configured to generate PPG data 102 by measuring changes in light absorption of blood vessels. Returning again to FIG. 1A, in one example, the accelerometer data 106 can be historical accelerometer data associated with a user of an accelerometer, or from accelerometers associated with a plurality of users. For example, accelerometer data generated by an accelerometer included in a user device (e.g., a fitness tracker, activity monitor, or the like) can be collected and stored, and the historical accelerometer data can be provided as input to the ANN model 104, as described below.

The PPG data 102 can be input to the ANN model 104 which generates a PPG heart rate probability 108 from the PPG data 102. For example, the PPG heart rate probability 108 may be a PPG heartbeat confidence score having a value between zero (0) and one (1). The PPG heartbeat confidence score can represent a probability that there is a heartbeat-induced fluctuation in the PPG data 102 at that sample. In one example, the accelerometer data 106 can be input to the same ANN model 104, or a portion of the same ANN model 104, which generates a motion-based heart rate probability 110 from the accelerometer data 106. For example, the motion-based heart rate probability 110 may produce or generate a motion-based heart rate confidence score having a value between zero (0) and one (1). The motion-based heart rate confidence score can represent a probability that there is a motion-induced fluctuation in the accelerometer data 106 at that sample that mimics a heart rate. The motion-based heart rate probability 110 output by the ANN model 104 can be evaluated to determine whether a motion-based signal in the accelerometer data 106 mimics a heart rate. More specifically, as in block 114, a motion-based signal can be provided to the heart rate selection module 112 when the motion-based heart rate probability 110 indicates a high-probability that the motion-based signal in the accelerometer data 106 simulates a heart rate. In the case of a low-probability that the motion-based signal in the accelerometer data 106 does not simulate a heart rate, then the motion-based signal is not provided to the heart rate selection module 112.

As in block 116, the heart rate selection module 112 can identify a PPG signal in the PPG data 102 that corresponds to a motion-based signal that mimics a true heart rate (as described in greater detail later) and, as in block 118, the heart rate selection module 112 can remove the PPG signal (as a heart rate candidate or from the PPG data 102). After removing the signal that corresponds to the motion-based signal, the heart rate selection module 112 can select a signal having the highest heart rate confidence score as the true heart rate, as in block 120. The heart rate selection module 112 can then provide the predicted heart rate along with the associated confidence score 122.

Referring again to FIG. 1B, the method described above can improve the performance of a device 130 to more accurately predict the heart rate of a user. As an example, the device 130 can be a fitness tracker, activity monitor, smart baby monitor, and the like configured with the ANN model 104 (which has been trained using PPG data) and the heart rate selection module 112. During use of the device 130, PPG data generated by the PPG sensor 132 can be input to the ANN model 104 to obtain a PPG heart rate probability (i.e., a PPG heart rate signal), and accelerometer data generated by the accelerometer 134 can be input to the ANN model 104 to obtain a motion-based heart rate probability (i.e., a motion-based signal that resembles a heart rate). The heart rate selection module 112 can identify a signal in the PPG data that corresponds to the motion-based heart rate signal and remove the signal (e.g., remove from consideration and/or remove from the PPG data 102) to improve the performance of the device 130 to accurately predict the heart rate of the user.

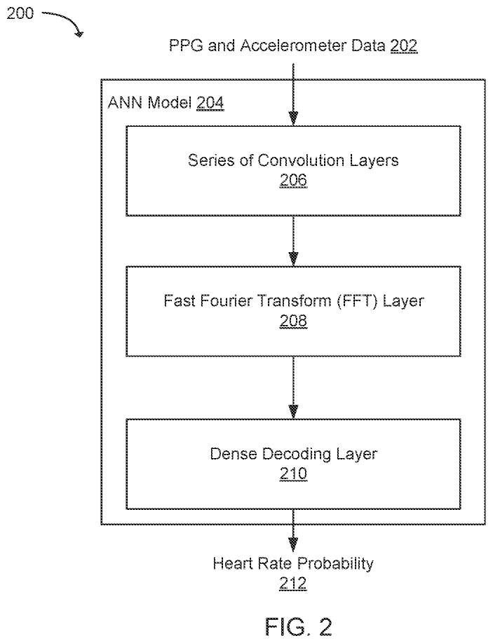

FIG. 2 is a block diagram illustrating a high-level example of a processing system 200 that includes an ANN model 204 used to generate an improved or corrected heart rate probability 212 using PPG and accelerometer data 202. The ANN model 204 can be trained using PPG data, and the ANN model 204 can be used to extract a predicted heart rate from both PPG and accelerometer input data 202. After training, the ANN model 204 can be put into a production environment to infer heart rate values. In pulse oximetry, there are typically two (and sometimes more) waveforms containing heart rate information, a red light signal with a peak wavelength around 660 nm and an infrared light signal with a peak wavelength around 940 nm. Using the red light and infrared light signals can strengthen the accuracy of heart rate predictions. These additional sources of input information (i.e., red and infrared light signals) can be incorporated into heart rate predictions at inference time to generate current heart rate predictions.

The ANN model 204, in one example, is an end-to-end neural network having an architecture that includes a series of convolution layers 206 followed by a fast Fourier transform (FFT) layer 208 and a dense decoding layer 210. As will be appreciated, the ANN model 204 can include additional components used to generate a heart rate probability. The PPG and accelerometer data 202 can be separately provided as input to the ANN model 204, and the architecture of the ANN model 204 can be configured to generate a heart rate probability 212. In one example, the accelerometer data can correspond to a time frame of the PPG data, such that the PPG data and the accelerometer data is captured during the same time frame (e.g., a ten, twenty, sixty, etc. time frame). In another example, a previously captured dataset of accelerometer data (for a specific user or from a plurality of users) can be used as input to the ANN model 204.

As illustrated, the architecture of the ANN model 204 includes a series of convolution layers 206. The series of convolution layers 206 can include any number of convolution layers. In a specific example of the architecture of the ANN model 204, the series of convolution layers 206 can include three convolution layers. In some examples, the series of convolution layers 206 may be a first convolution layer that proceeds the FFT layer 208, and the architecture of the ANN model 204 can include a second series of convolution layers (not shown) located between the FFT layer 208 and the dense decoding layer 210. The second series of convolution layers can be used to identify and remove artifacts from a Fourier transform output by the FFT layer 208.

The convolution layers 206 of the PPG data trained ANN model 204 can be configured to identify a PPG signal in PPG data and an acceleration signal in accelerometer data. The PPG data can be obtained from a PPG sensor, such as a heart rate monitor device or pulse oximeter monitor device. A PPG is an optically obtained plethysmogram used to detect blood volume changes in the microvascular bed of tissue of a subject. A PPG sensor illuminates the skin and measures changes in light absorption to monitor the perfusion of blood to the dermis and subcutaneous tissue of the skin. The PPG sensor detects a change in blood volume and measures an amount of light either transmitted or reflected to a photodiode. The PPG sensor generates PPG data containing a PPG signal or PPG waveform where each cardiac cycle appears as a peak in the PPG signal. Accelerometer data can be obtained from an accelerometer device. The accelerometer device measures proper acceleration by detecting vibration, and can in some cases, detect both the magnitude and the direction of the proper acceleration. The convolution layers 206 of the PPG data trained ANN model 204 can analyze the PPG and accelerometer data 202 obtained from the PPG sensor and accelerometer to identify signals that correspond to heart rates.

The architecture of the ANN model 204 shown in FIG. 2 places the FFT layer 208 between the convolution layer 206 and the dense decoding layer 210. The FFT layer 208 can be configured to apply a fast Fourier transform to a signal (i.e., a PPG signal and acceleration signal) output by the series of convolution layers 206 to convert the signal to a representation of a fundamental frequency and harmonic frequencies. Applying a fast Fourier transform to a signal allows resulting frequencies to be quantized into values that can be classified into heart rate values.

Placing the FFT layer 208 after the series of convolution layers 206 and before the dense decoding layer 210 improves performance of predicting heart rates using the ANN model 204. In particular, applying a fast Fourier transform to a signal output by the series of convolution layers 206 reduces a number of parameters that are provided to the dense decoding layer 210 of the ANN model 204. By reducing the number of parameters provided as input to the dense decoding layer 210, an amount of data processed by the dense decoding layer 210 is decreased, which results in a shorter amount of time to generate heart rate probabilities 212.

Also, placing the FFT layer 208 after the series of convolution layers 206 and before the dense decoding layer 210 improves accuracy of heart rate predictions output by the ANN model 204. More specifically, applying a fast Fourier transform to signals output by the series of convolution layers 206 allows frequencies to be quantized, thereby restricting a number of possible frequency values that can be classified as heart rate values. The signals output by the FFT layer 208 provide signal representations that are easier to decode and increase the accuracy of a heart rate prediction as compared to using alternative techniques. For example, using the alternative technique of a mean squared error function as a loss function tends to pull values toward the mean, which creates bias in PPG and accelerometer data 202. Using a fast Fourier transform technique reduces the chance of this bias. For example, a fast Fourier transform technique allows frequencies output by the FFT layer 208 to be classified as a probability distribution of heart rates, and allows for a maximum likelihood estimation to be applied to the probability distribution of heart rates to determine a heart rate probability 212.

The dense decoding layer 210 included in the ANN model 204 architecture can be configured to decode frequency representations output by the FFT layer 208 into heart rate predictions. In one example, the dense decoding layer 210 decodes the frequency representations into heart rate information (e.g., beats per minute (BPM)) used to generate a heart rate prediction. As an example, the dense decoding layer 210 selects a frequency representation (e.g., a harmonic frequency) output by the FFT layer 208 and applies a mask to the PPG frequency representation which is used to visualize the frequency representation as a heart rate value (e.g., 131, 132, or 133 BPM). Thereafter, the heart rate values can be scored to create a probability distribution that indicates a maximum likelihood of a heart rate, which can be output as a heart rate probability 212. In one example, after scoring the heart rate values, the heart rate values can be input to a softmax layer (not shown) that has one neuron for each heart rate value. The softmax layer can normalize the heart rate values to sum to a value of one (1), creating a probability distribution of heart rate values that indicates a maximum likelihood of a heart rate value.

The following example is an illustration of an end-to-end ANN architecture configured to generate heart rate probabilities based on PPG and accelerometer data input. As will be appreciated, the example artificial neural network architecture shown in Example 2 is merely representative of an ANN architecture and is not meant to be limiting.

The ANN model 204 can be trained to generate heart rate probabilities using a training dataset of PPG data. The training data set can comprise PPG data collected from subjects using a PPG sensor. PPG data can then be split into a training dataset and a test dataset. In some examples, synthetic PPG data can be generated to supplement the training dataset. For example, synthetic PPG data can be generated to have a uniform heart rate that is between 30-300 beats per minute (BPM). Also, additional synthetic PPG data containing noise and no PPG signal can be added to the training dataset to train the ANN model 204 to indicate an uncertainty of a true heart rate. As an example, synthetic PPG data can be labeled with a zero (0) heart rate to correspond to an unknown value. In one example, the ANN model 204 can be trained using categorical cross entropy to label PPG data in the training dataset to a heart rate category and an Adam optimizer to update weights assigned to the PPG data. As will be appreciated, techniques other than those described above can be used to train the ANN model 204.

The various processes and/or other functionality described above may be executed on one or more processors that are in communication with one or more memory modules. The processing system 200 may include one or more computing devices. In some examples, the processing system 200 can include a plurality of data stores used to store PPG and accelerometer data 202 and/or heart rate probabilities 212 output by the PPG data trained ANN model 204. The term “data store” may refer to any device or combination of devices capable of storing, accessing, organizing and/or retrieving data. The storage system components of the data store may include storage systems such as volatile or non-volatile RAM, hard-drive type media, and a cloud storage network. The data store may be representative of a plurality of data stores as can be appreciated.

In some examples, the processing system 200 may include a network for transmitting data between servers, clients, and devices. The network may include any useful computing network, including an intranet, the Internet, a local area network, a wide area network, a wireless data network, or any other such network or combination thereof. Components utilized for such a system may depend at least in part upon the type of network and/or environment selected. Communication over the network may be enabled by wired or wireless connections and combinations thereof.

FIG. 2 illustrates that certain processing modules may be discussed in connection with this technology, and these processing modules may be implemented as computing services. In one example configuration, a module may be considered a service with one or more processes executing on a server or other computer hardware. Such services may be centrally hosted functionality or a service application that may receive requests and provide output to other services or consumer devices. For example, modules providing services may be considered on-demand computing that are hosted in a server, virtualized service environment, service provider environment (e.g., cloud environment), grid or cluster computing system. An API may be provided for each module to enable a second module to send requests to and receive output from the first module. Such APIs may also allow third parties to interface with the module and make requests and receive output from the modules. While FIG. 2 illustrates an example of a system that may implement the techniques above, many other similar or different environments are possible. The example environments discussed and illustrated above are merely representative and not limiting.

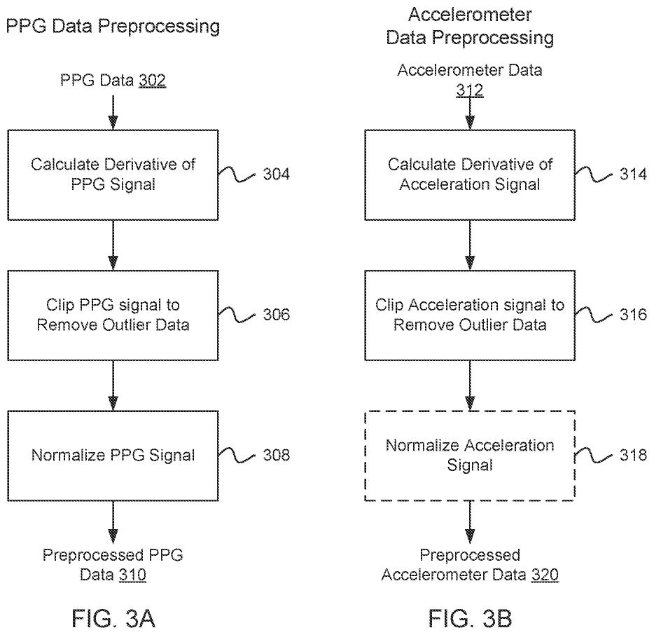

FIGS. 3A-B are flow diagrams that illustrate example methods for preprocessing PPG data 302 and accelerometer data 312. Prior to inputting PPG data 302 and/or accelerometer data 312 to the ANN model 204 described above, the data 302/312 can be preprocessed to format the data 302/312 for input to the ANN model 204. Preprocessing can be performed prior to training the ANN model 204 and prior to inference time in which a predicted heart rate value is generated.

Preprocessing of data 302/312 can include one or more preprocessing steps. In one example, the preprocessing steps can include: (i) calculating a derivative of a signal to accentuate high frequency components in the signal, (ii) clipping the signal to remove outlier data included in the data 302/312, and (iii) normalizing the signal to a standard deviation.

Referring to FIG. 3A, as in block 304, the preprocessing step of taking a derivative of a PPG signal can be performed to accentuate high frequency components in the PPG signal and diminish the effects of lower frequency motion artifacts. As in block 306, the preprocessing step of clipping the PPG signal can be performed to remove outlier data included in the PPG data 302. Outliers can be caused by user movement and clipping can reduce the influence of the outliers in the PPG signal. In one example, clipping the PPG signal can include computing amplitude percentiles of the PPG signal and clipping values that are greater than a difference between amplitude percentiles. As an illustration, the 25th, 50th, and 75th amplitude percentiles of a PPG signal can be computed, and values can be clipped that are greater than six (6) times the difference between the 50th and 75th percentile of less than six (6) times the difference between the 50th percentile and the 25th percentile. As in block 308, the preprocessing step of normalizing the PPG signal to a standard deviation can be performed to ensure that PPG signals included in PPG data 302 are approximately the same scale. After preprocessing the PPG data 302, the preprocessed PPG data 310 can be provided as input to the ANN model 204 to generate heart rate probabilities as described earlier.

Now referring to FIG. 3B, as in block 314, the preprocessing step of taking a derivative of an acceleration signal can be performed to accentuate high frequency components in the acceleration signal and diminish the effects of lower frequency artifacts. As in block 316, the preprocessing step of clipping the acceleration signal can be performed to remove outlier data included in the acceleration data 312. At this point, no further preprocessing may be needed and the preprocessed acceleration data 320 can be provided as input to the ANN model 204 shown in FIG. 2. That is, in this example, normalization of the acceleration data 312 may not be performed. In another example, as in block 318 (shown in dashed line to indicate an optional step), the preprocessing step of normalizing the acceleration signal to the standard deviation, as used to normalize the PPG signal described above, can be performed to ensure that acceleration signals included in acceleration data 312 are approximately the same scale used for the PPG signal. After preprocessing the acceleration data 312, the preprocessed acceleration data 320 can be provided as input to the ANN model 204 to classify the acceleration signal as a heart rate probability used to identify a motion-based signal in the PPG signal, which can be removed (ignored) to improve or correct the accuracy of a heart rate prediction.

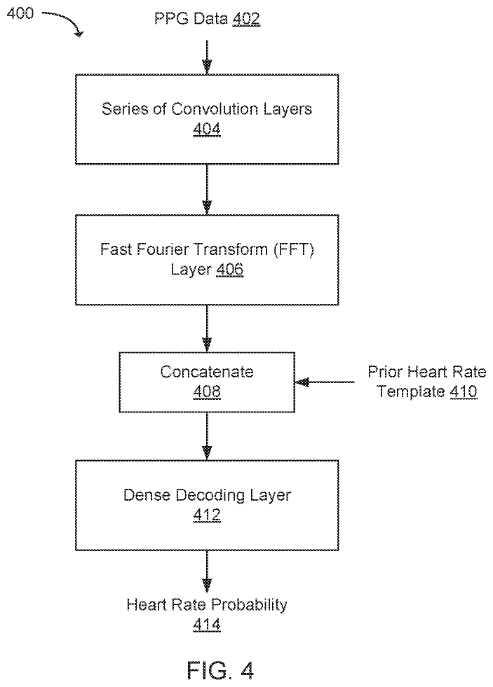

FIG. 4 is a block diagram that illustrates an example network architecture for an artificial ANN model 400 which incorporates prior heart rate information to generate a heart rate probability 414. The network architecture shown in FIG. 4 incorporates prior heart rate information into the ANN model 400 to allow the prior heart rate information to be used in training of the ANN model 400.

Prior predictions of heart rates can be used as part of generating a current heart rate probability in a number of ways. In one example, a series of sine waves corresponding to a fundamental frequency and harmonic frequencies of a prior heart rate prediction can be summed. The resulting sum provides a prior heart rate template 410 which can be passed to the ANN model 400. One method that can be used to pass a prior heart rate template 410 to the ANN model 400 includes concatenating a Fourier transform of the prior heart rate template 410 to the Fourier transform output by the FFT layer 406 of the ANN model 400. As an illustration, PPG data 402 included in a training dataset can be input to a series of convolution layers 404 to remove artifacts and clean up the PPG signal. The FFT layer 406 can be applied to the PPG signal to produce a Fourier transform of the PPG signal. A Fourier transform of a prior heart rate template 410 can be produced, and the Fourier transform of a prior heart rate template 410 can be concatenated 408 to the Fourier transform of the PPG signal. The resulting concatenated Fourier transform, comprising PPG frequency representations of the PPG data 402 and the prior heart rate template 410, can be input to a dense decoding layer 412 of the ANN model 400. The dense decoding layer 412 decodes the PPG frequency representations, as described earlier, and outputs a heart rate probability 414.

The following example illustrates an end-to-end ANN architecture configured to incorporate prior heart rate information into an artificial ANN model to generate a heart rate probability. As will be appreciated, the example artificial neural network architecture shown in Example 2 is merely representative of an ANN architecture and is not limiting.

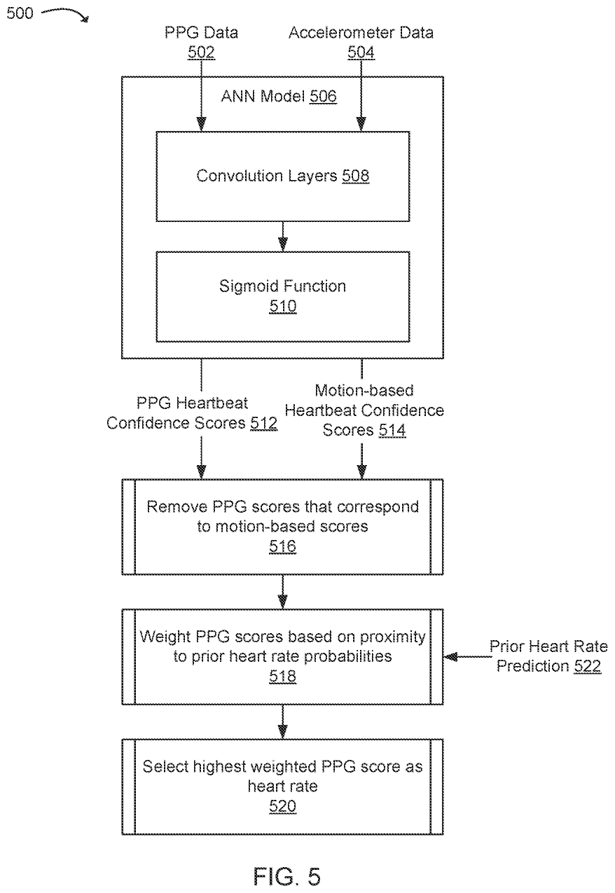

FIG. 5 is a block diagram that illustrates a system 500 containing an example ANN model 506 configured to generate heartbeat confidence scores (e.g., peak probabilities) that correspond to individual heart rate peaks, which can be decoded and compared to prior heart rate probabilities 522 to produce a final heart rate prediction. In one example, heartbeat confidence scores 512/514 can be decoded using peak detection to produce a final heart rate probability. In one example, a peak detection method can use the preprocessing technique described above in association with FIGS. 3A-B to preprocess PPG data 502 and accelerometer data 504. Following preprocessing, a signal can be passed to the ANN model 506 in defined windows (e.g., 100, 125, or 150) of samples of a PPG signal and/or acceleration signal. The ANN model 506 can include a series of convolution layers 508 with a sigmoid function 510 output corresponding to a heartbeat confidence scores 512/514. Illustratively, the sigmoid function 510 can map each data point to be between zero (0) and one (1). The sigmoid function 510 can serve a similar purpose as the RELU function in example 3 below, with the added behavior of bounding the output.

Example 3 below illustrates an ANN architecture configured to generate a heartbeat confidence score. As will be appreciated, the example ANN architecture shown in Example 3 is merely representative and is not meant to be limiting.

After generating heartbeat confidence scores 512/514 from the PPG data 502 and the accelerometer data 504, a heart rate selection method can be used to identify and remove a motion-based signal from consideration as a true heart rate and/or remove the motion-based signal from the PPG data. For example, as in block 516, it is believed that the heart rate selection method can remove the motion-based signal from the PPG data that corresponds to the accelerometer heartbeat confidence scores.

In the example illustrated in FIG. 5, the PPG heartbeat confidence scores can be weighted based on proximity to prior heart rates. For example, as in block 518, one or more prior heart rate probabilities 522 for prior heart rate predictions can be obtained, and a proximity of a prior heart rate probability 522 can be compared to a PPG heartbeat confidence score 512. A weight can be assigned to the PPG heartbeat confidence score512. The weight may be based on a proximity of a prior heart rate probability 522 to the PPG heartbeat confidence score 512. The weight can be used to indicate a likelihood that the PPG heartbeat confidence score 512 is accurate and not an anomaly in the dataset. After assigning weights to the PPG heartbeat confidence scores 512, the frequency of the highest weighted PPG heartbeat confidence scores can be selected as the heart rate prediction, as in block 520.

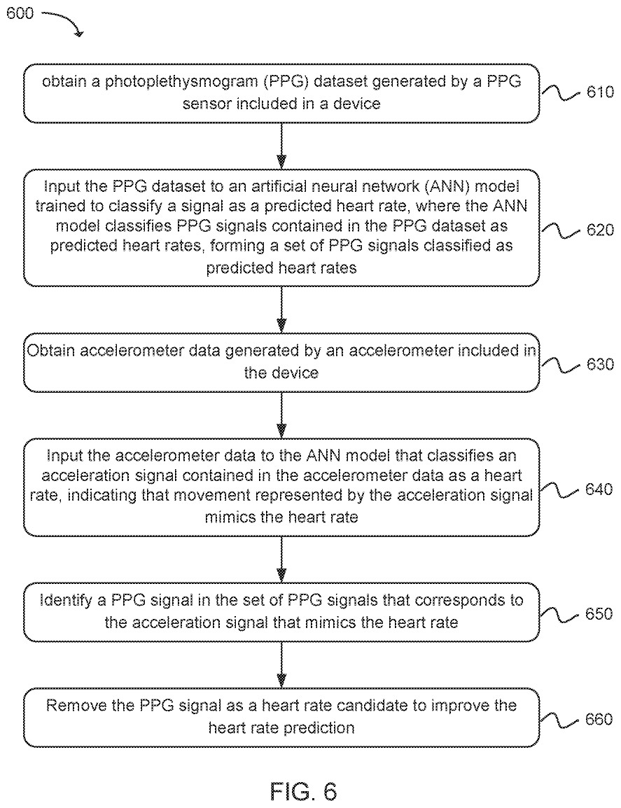

FIG. 6 is a flow diagram illustrating an example method 600 for improving the accuracy of a heart rate prediction by identifying and removing a motion-based signal as a heart rate candidate. Beginning in block 610, a PPG dataset generated by a PPG sensor included in a device (e.g., a fitness tracker, health monitor, etc.) can be obtained, and as in block 620, the PPG dataset can be input to an ANN model trained to classify a signal as a predicted heart rate. The ANN model can be trained to predict a heart rate using a training dataset of PPG data, and the ANN model can accept data from a plurality of sources (e.g., PPG sensor, accelerometer, and other types of sensors) and determine whether a signal in the data corresponds to a heart rate. In one example, the ANN model can include a first series of convolution layers to identify a PPG signal in PPG data and remove artifacts contained in the PPG data, a fast Fourier transform (FFT) layer to convert the PPG signal to PPG frequency representations, and a dense layer to decode the PPG frequency representations to heart rate predictions. In one example, an output layer of the ANN model is a softmax layer that has a neuron node for each heart rate value, and the softmax layer can output a heart rate probability map that provides a probability for each heart rate value in a PPG dataset. In one example, prior to training the ANN model using the training dataset, the PPG data contained in the training dataset can be preprocessed. Preprocessing the PPG data can include (i) calculating a derivative of the PPG signal to accentuate high frequency components, (ii) clipping the PPG signal to remove outlier data included in the PPG data, and (iii) normalizing a PPG signal to a standard deviation. In one example, the ANN model can be trained using categorical cross entropy to label the PPG data in the training dataset to a heart rate category and an Adam optimizer to update weights assigned to the PPG data. In some examples, prior heart rate information can be used to train the ANN model. For example, a prior heart rate template can be generated by summing a series of sine waves that correspond to a fundamental frequency of a prior heart rate prediction and a harmonic of the prior heart rate prediction, and the prior heart rate template can be input to the ANN model during training of the ANN model. Inputting a prior heart rate prediction to the ANN model during training can include (i) calculating a Fourier transform of a prior heart rate prediction, (ii) concatenating the Fourier transform of the prior heart rate prediction to a Fourier transform output by the FFT layer to form a concatenated Fourier transform, and (iii) providing the concatenated Fourier transform to the dense layer of the ANN model.

One or more PPG signals contained in the PPG dataset input to the ANN model can be classified as predicted heart rates. The method 600 can be used to correct a heart rate prediction by identifying a motion-based signal included in the PPG dataset and removing the motion-based signal. More specifically, as in block 630, accelerometer data generated by an accelerometer included in the device can be obtained, and as in block 640, the accelerometer data can be input to the ANN model, which classifies an acceleration signal contained in the accelerometer data as a heart rate, thereby indicating that movement represented by the acceleration signal mimics a heart rate. Thereafter, as in block 650, a PPG signal included in the PPG dataset can be identified as corresponding to the acceleration signal that mimics the heart rate, and as in block 660, the PPG signal can be removed from consideration as a heart rate probability and/or removed from the PPG dataset to correct the heart rate prediction.

In one example configuration, a prior heart rate prediction (e.g., an immediate prior heart rate prediction or a prior heart rate prediction within a defined time frame) output by the artificial ANN model can be obtained, and the prior heart rate prediction can be compared to a current heart rate prediction output by the artificial ANN model to determine whether the current heart rate prediction is within a quality threshold of the prior heart rate prediction. For example, the prior heart rate prediction can be compared to the current heart rate prediction by (i) generating a heart rate distribution, wherein the current heart rate prediction is multiplied by a Gaussian function that has a mean value equal to the prior heart rate prediction, (ii) calculating an argmax of the heart rate distribution to produce the heart rate prediction, and/or (iii) accept the heart rate prediction if confidence score is greater than a quality threshold. In the case that a prior heart rate prediction is unavailable, a heart rate distribution can be generated by multiplying a current heart rate prediction by an identity vector, and calculating an argmax of the heart rate distribution to produce the heart rate prediction.

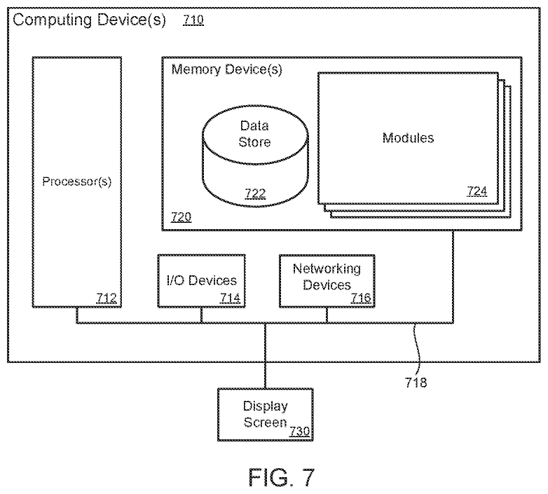

FIG. 7 illustrates a computing device 710 on which modules of this technology may execute. The computing device 710 may include one or more processors 712 that are in communication with one or more memory devices 720. The computing device 710 may include a local communication interface 717 for the components in the computing device 710. For example, the local communication interface 718 may be a local data bus and/or any related address or control busses as may be desired.

A memory device 720 may contain modules 724 that are executable by the processor(s) 712 and data for the modules 724. The modules 724 can include ANN modules, convolution modules, fast Fourier transform modules, dense decoding modules, and other modules. The modules 724 may execute the functions described earlier. A data store 722 may also be located in the memory device 720 for storing data related to the modules 724 and other applications along with an operating system that is executable by the processor(s) 712.

Other applications may also be stored in the memory device 720 and may be executable by the processor(s) 712. Components or modules discussed in this description may be implemented in the form of software using high-level programming languages that are compiled, interpreted, or executed using a hybrid of the methods.

The computing device 710 may also have access to I/O (input/output) devices 714 that are usable by the computing device 710. One example of an I/O device is a display screen 730 that is accessible to the computing device 710. Networking devices 716 and similar communication devices may be included in the computing device 710. The networking devices 716 may be wired or wireless networking devices that connect to the internet, a LAN, WAN, or other computing network.

The components or modules that are shown as being stored in the memory device 720 may be executed by the processor(s) 712. The term “executable” may mean a program file that is in a form that may be executed by a processor 712. For example, a program in a higher level language may be compiled into machine code in a format that may be loaded into a random access portion of the memory device 720 and executed by the processor 712, or source code may be loaded by another executable program and interpreted to generate instructions in a random access portion of the memory device 720 to be executed by the processor(s) 712. The executable program may be stored in any portion or component of the memory device 720. For example, the memory device 720 may be random access memory (RAM), read only memory (ROM), flash memory, a solid state drive, memory card, a hard drive, optical disk, floppy disk, magnetic tape, or any other memory components.

The processor(s) 712 may represent multiple processors and the memory device 720 may represent multiple memory units that operate in parallel to the processing circuits. This may provide parallel processing channels for the processes and data in the computing device 710. The local communication interface 718 may be used as a network to facilitate communication between any of the multiple processors and multiple memories. The local communication interface 718 may use additional systems designed for coordinating communication such as load balancing, bulk data transfer, and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting, or for similar reasons.

Some of the functional units described in this specification have been labeled as modules in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media, implemented with any technology for the storage of information such as computer readable instructions, data structures, program modules, or other data. Computer readable storage media includes, but is not limited to, a non-transitory machine readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

1. A system for improving a heart rate prediction, comprising:
at least one processor;
a memory device including instructions that, when executed by the at least one processor, cause the system to:
obtain a photoplethysmogram (PPG) dataset generated by a PPG sensor included in a device;
input the PPG dataset to a machine learning model to classify a PPG signal in the PPG dataset as a predicted heart rate;
obtain accelerometer data generated by an accelerometer included in the device;
input the accelerometer data to the machine learning model to classify an acceleration signal contained in the accelerometer data as a heart rate, wherein movement represented by the acceleration signal mimics the heart rate;
identify the PPG signal in the PPG dataset as corresponding to the acceleration signal that mimics the heart rate; and
remove the PPG signal that corresponds to the acceleration signal as a heart rate candidate to improve accuracy of the heart rate prediction.

2. The system of claim 1, wherein the instructions to remove the PPG signal further cause the system to:
remove PPG signal in the PPG dataset that corresponds to the acceleration signal that mimics the heart rate; and
select a remaining PPG signal in the PPG dataset as the heart rate prediction.

3. The system of claim 1, wherein the memory device further includes instructions that, when executed by the at least one processor, cause the system to:
assign weights to PPG heartbeat confidence scores output by the machine learning model based on a proximity of a PPG heartbeat confidence score to a prior heart rate prediction; and
select a highest weighted PPG heartbeat confidence score as the heart rate prediction.

4. The system of claim 1, wherein the memory device further includes instructions that, when executed by the at least one processor, cause the system to compare the heart rate prediction to a prior heart rate prediction to determine whether the heart rate prediction is within a quality threshold of the prior heart rate prediction.

5. The system of claim 1, wherein the machine learning model is an artificial neural network (ANN) model.

6. The system of claim 5, wherein the ANN model includes a first series of convolution layers to identify the PPG signal in the PPG dataset and remove artifacts contained in the PPG dataset, a fast Fourier transform (FFT) layer used to identify PPG frequencies in the PPG dataset, and a dense layer used to decode a heart rate value from the PPG frequencies.

7. The system of claim 6, wherein the FFT layer is located after the first series of convolution layers to identify fundamental and harmonic frequencies of the PPG signal.

8. The system of claim 6, wherein the ANN model includes a second series of convolution layers located between the FFT layer and the dense decoding layer, wherein the second series of convolution layers identify and remove artifacts from a Fourier transform output by the FFT layer.

9. The system of claim 5, wherein an output layer of the ANN model is a softmax layer that has a neuron node for each heart rate value.

10. The system of claim 1, wherein the device is a wearable device.

11. A computer implemented method for improving a heart rate prediction, comprising:
receiving a photoplethysmogram (PPG) dataset generated by a PPG sensor;
inputting the PPG dataset to a machine learning model to classify a PPG signal in the PPG dataset as a predicted heart rate, wherein the machine learning model has been trained using PPG training data to classify a signal from a generic data source as a heart rate;
receiving accelerometer data generated by an accelerometer;
inputting the accelerometer data to the machine learning model to classify an acceleration signal contained in the accelerometer data as a heart rate, wherein movement represented by the acceleration signal mimics the heart rate;
identifying a PPG heartbeat confidence score for the PPG signal that corresponds to the acceleration signal that mimics the heart rate; and
removing the PPG heartbeat confidence score as a heart rate candidate to improve the accuracy of the heart rate prediction.

12. The computer implemented method of claim 11, further comprising training the machine learning model using categorical cross entropy to label PPG data in the PPG training data to a heart rate category and an Adam optimizer to update weights assigned to the PPG data.

13. The computer implemented method of claim 11, wherein the accelerometer data corresponds to a time frame of the PPG dataset.

14. The computer implemented method of claim 11, wherein the accelerometer data is historical accelerometer data associated with a user of the accelerometer.

15. The computer implemented method of claim 11, wherein the accelerometer data is historical accelerometer data obtained from accelerometers associated with a plurality of users.

16. The computer implemented method of claim 11, wherein the machine learning model includes a series of convolution layers with a sigmoid function to produce an output corresponding to a heart rate confidence score.

17. The computer implemented method of claim 11, further comprising:
assigning weights to PPG heartbeat confidence scores based on a proximity of a PPG heartbeat confidence score to a prior heart rate confidence score; and
selecting a PPG signal as the heart rate prediction based on a proximity of the PPG heartbeat confidence score to the prior heart rate confidence score.

18. A non-transitory machine-readable storage medium including instructions embodied thereon, wherein the instructions, when executed by at least one processor:
receive a photoplethysmogram (PPG) dataset generated by a PPG sensor;
input the PPG dataset to a machine learning model to classify a PPG signal in the PPG dataset as a heart rate prediction, wherein a PPG heartbeat confidence score is assigned to the PPG signal;
receive accelerometer data generated by an accelerometer, wherein the accelerometer data corresponds to a time frame of the PPG dataset;
input the accelerometer data to the machine learning model to classify an acceleration signal contained in the accelerometer data as a heart rate, wherein a motion-based heart rate confidence score is assigned to the acceleration signal, and wherein movement represented by the acceleration signal mimics the heart rate;
identify a PPG heartbeat confidence score that corresponds to the motion-based heart rate confidence score; and
remove the PPG signal associated with the PPG heartbeat confidence score as a heart rate candidate to improve accuracy of the heart rate prediction.

19. The non-transitory machine-readable storage medium in claim 17, wherein the instructions, when executed by the at least one processor, further input defined windows of samples comprising the PPG signal and the acceleration signal to the machine learning model.

20. The non-transitory machine-readable storage medium in claim 17, wherein the instructions that remove the PPG signal further:
remove the PPG signal associated with the PPG heartbeat confidence score that corresponds to the acceleration heart rate confidence score from the PPG dataset.
